(19) United States
(12) Patent Application Publication
(10) Pub. No.: US 2024/0042177 A1
(43) Pub. Date: Feb. 8, 2024

(54) GUIDE WIRE

(71) Applicant: ASAHI INTECC CO., LTD., Seto-shi (JP)

(72) Inventors: Soichi FUTAMI, Seto-shi (JP); Masakazu NAKADA, Seto-shi (JP); Junji SATOZAKI, Seto-shi (JP); Yumi TAKATA, Seto-shi (JP)

(73) Assignee: ASAHI INTECC CO., LTD., Seto-shi (JP)

(21) Appl. No.: 18/379,279

(22) Filed: Oct. 12, 2023

Related U.S. Application Data
(63) Continuation of application No. PCT/JP2022/018007, filed on Apr. 18, 2022.

(30) Foreign Application Priority Data
Apr. 30, 2021 (JP) 2021-077038

(51) Int. Cl. A61M 25/09

(52) U.S. Cl. CPC A61M 25/09; A61M 2025/09133; A61M 2025/09191

(57) ABSTRACT

The guide wire includes a core shaft, a coil body with a wire helically wound around the outer periphery of the core shaft, and a coating layer provided on the outer periphery of the coil body. The coating layer has a peak portion protruding in the outer peripheral direction of the guide wire in a swollen state. On a cross-section including a central axis of the coil body, an apex of the peak portion is present at a position on a proximal end side of a first virtual straight line that extends through a central point of a first transverse section of the wire and is perpendicular to the central axis and at a position on a distal end side of a second virtual straight line that extends through a central point of second transverse section of the wire and is perpendicular to the central axis.

### CROSS-REFERENCE TO RELATED APPLICATIONS

This is a Continuation of Application No. PCT/JP2022/018007 filed Apr. 18, 2022, which claims priority to JP 2021-077038 filed Apr. 30, 2021. The disclosure of the prior applications is hereby incorporated by reference herein in its entirety.

### TECHNICAL FIELD

The technique disclosed in the present specification relates to a guide wire.

### BACKGROUND

A guide wire is used to guide a medical device (hereinafter, referred to as a “combined device”) such as a catheter to a predetermined position inside a human body. The guide wire includes a core shaft and a coil body in which a wire is helically wound around the outer periphery of the core shaft.

A hydrophilic coating layer is provided on the outer periphery of the coil body in order to improve lubricity of the guide wire. The coating layer absorbs moisture inside a human body and swells. When a combined device guided by the guide wire is pressed against the coating layer of the guide wire at a bent portion or the like of the combined device, the coating layer is crushed to have a smooth surface, and moisture is extruded from the coating layer to form a thin moisture membrane between the smooth surface of the coating layer and the combined device; as a result, lubricity between the guide wire and the combined device is ensured.

Conventionally, a technique of making the shape of a surface of the coating layer covering a base material layer have irregularities in order to improve lubricity of a medical device has been known (see Patent Literature 1, for example).

### CITATION LIST

### Patent Literature

Patent Literature 1: Japanese Unexamined Patent Application Publication No. 2013-146504

### SUMMARY

### Technical Problem

In the conventional guide wire, when a combined device is pressed against the coating layer, the coating layer is crushed, and the combined device comes into contact with the wire of the coil body, possibly causing decrease in lubricity of the guide wire. Note that, the combined device cannot be prevented from coming into contact with the wire of the coil body only by making the shape of the surface of the coating layer have irregularities without considering the positional relation with the coil body as in the conventional technique, still possibly causing decrease in lubricity of the guide wire. It can be also considered that the thickness of the coating layer is increased in order to avoid contact between the combined device and the wire of the coil body; however, merely by increasing the thickness of the coating layer, the thickness of the coating layer becomes excessively large when swelling, possibly causing deterioration in passability of the combined device. As described above, there is room for improvement regarding achievement of both lubricity and passability of a combined device in conventional guide wires.

The present specification discloses a technique capable of solving the above-described problem.

### Solution to Problem

The technique disclosed herein can be implemented, for example, as the following aspects.
(1) A first guide wire disclosed herein includes a core shaft, a coil body in which a wire is helically wound around the outer periphery of the core shaft, and a coating layer provided on the outer periphery of the coil body. The coating layer is configured to have a peak portion protruding in the outer peripheral direction of the guide wire in a swollen state. On a cross-section including the central axis of the coil body, an apex of the peak portion is present at a position on the more proximal end side than a first virtual straight line passing the central point of one transverse section of the wire and perpendicular to the central axis and at a position on the more distal end side than a second virtual straight line passing the central point of another transverse section of the wire and perpendicular to the central axis, the another transverse section being adjacent to and on the proximal end side of the one transverse section.

As described above, since the coating layer of the present guide wire is configured to have the peak portion protruding in the outer peripheral direction of the guide wire in the swollen state, the peak portion in the coating layer effectively exhibits a water retention function, and high lubricity is imparted to the guide wire. In the present guide wire, on a cross-section including the central axis of the coil body, an apex of the peak portion of the coating layer in the swollen state is present at a position on the more proximal end side than the first virtual straight line passing the central point of one transverse section of the wire and perpendicular to the central axis and at a position on the more distal end side than the second virtual straight line passing the central point of another transverse section of the wire and perpendicular to the central axis, the another transverse section being adjacent to and on the proximal end side of the one transverse section. That is, the apex of the peak portion of the coating layer in the swollen state is positioned between coils of the wire of the coil body. The portion of the coating layer positioned between coils of the wire is a portion with large “allowance” when receiving stress from the outer peripheral side, since the presence of the wire as a base is minor therein. Therefore, when the apex of the peak portion is present in the above-described portion of the coating layer, stress from a combined device can be dispersed. Accordingly, according to the present guide wire, a combined device can be effectively prevented from coming into contact with the wire of the coil body even when the coating layer is crushed. In addition, the portion of the coating layer positioned between coils of the wire easy moves since the presence of the wire as a base is minor therein; therefore, passability of a combined device can be successfully maintained even when the apex of the peak portion of the coating layer is positioned between coils of the wire of the coil body. In view of the above, according to the present guide wire, both lubricity and passability of a combined device can be achieved at a high level.
(2) The above guide wire may be configured such that, on the cross-section including the central axis of the coil body, the apex of the peak portion may be present at a position not overlapping transverse sections of the wire in the direction perpendicular to the central axis. The portion of the coating layer present at a position not overlapping transverse sections of the wire of the coil body in the direction perpendicular to the central axis is a portion with significantly large “allowance” when receiving stress from the outer peripheral side, since the presence of the wire as a base is significantly minor therein. Therefore, when the apex of the peak portion is present in the above-described portion of the coating layer, stress from a combined device can be effectively dispersed. Accordingly, according to the present guide wire, a combined device can be effectively prevented from coming into contact with the wire of the coil body even when the coating layer is crushed.
(3) A second guide wire disclosed herein includes a core shaft, a coil body in which a wire is helically wound around the outer periphery of the core shaft, and a coating layer provided on the outer periphery of the coil body. The coating layer includes a first coating layer provided on the outer periphery of the coil body, and a second coating layer provided on the outer periphery of the first coating layer and having swellability higher than that of the first coating layer. On the cross-section including the central axis of the coil body, the thickness of the second coating layer on a perpendicular bisector of a virtual line segment connecting a first central point, which is the central point of one transverse section of the wire, and a second central point, which is the central point of another transverse section of the wire, is thicker than the thickness of the second coating layer on a first virtual straight line passing the first central point and perpendicular to the central axis and is thicker than the thickness of the second coating layer on a second virtual straight line passing the second central point and perpendicular to the central axis, the another transverse section being adjacent to and on the proximal end side of the one transverse section.

As described above, in the present guide wire, on the cross-section including the central axis of the coil body, the thickness of the second coating layer on the perpendicular bisector of the virtual line segment connecting the first central point, which is the central point of one transverse section of the wire, and the second central point, which is the central point of another transverse section of the wire, is relatively thick, the another transverse section being adjacent to and on the proximal end side of the one transverse section. Therefore, according to the present guide wire, the configuration in which the apex of the peak portion of the coating layer in a swollen state is present at a position on the more proximal end side than the first virtual straight line and at a position on the more distal end side than the second virtual straight line can be realized. Accordingly, according to the present guide wire, both lubricity and passability of a combined device can be achieved at a high level.
(4) The guide wire described above may be configured such that, on the cross-section including the central axis of the coil body, the thickness of the second coating layer on the perpendicular bisector is 1.2 times or more the thickness of the second coating layer on the first virtual straight line and is 1.2 times or more the thickness of the second coating layer on the second virtual straight line. According to the present guide wire, the configuration in which in the coating layer in the swollen state, the thickness of the portion positioned between coils of the wire of the coil body is thicker than the thickness of the portion positioned immediately above the wire by a certain factor or more can be realized, and both lubricity and passability of a combined device can be achieved at a higher level.

The technique disclosed herein can be realized in various aspects, for example, in an aspect of a guide wire, a method of producing same, and the like.

### BRIEF DESCRIPTION OF DRAWINGS

FIG. 1 is an explanatory diagram schematically illustrating a configuration of a guide wire 100 in the present embodiment.

FIG. 2 is an explanatory diagram illustrating a detailed configuration of a coating layer 30 of the present embodiment.

FIG. 3 is an explanatory diagram illustrating a detailed configuration of a coating layer 30 of the present embodiment.

FIG. 4 is an explanatory diagram illustrating a state of the guide wire 100 of the present embodiment during use.

FIG. 5 is an explanatory diagram illustrating a configuration of a coating layer in a guide wire 100X of a comparative example.

FIG. 6 is an explanatory diagram illustrating a configuration of the coating layer in the guide wire 100X of the comparative example.

FIG. 7 is an explanatory diagram illustrating a state of the guide wire 100X of the comparative example during use.

FIG. 8 is an explanatory diagram illustrating a swollen film thickness measurement result of a guide wire 100 of Example 1.

### DETAILED DESCRIPTION

### A. Embodiment

### A-1. Configuration of Guide Wire 100:

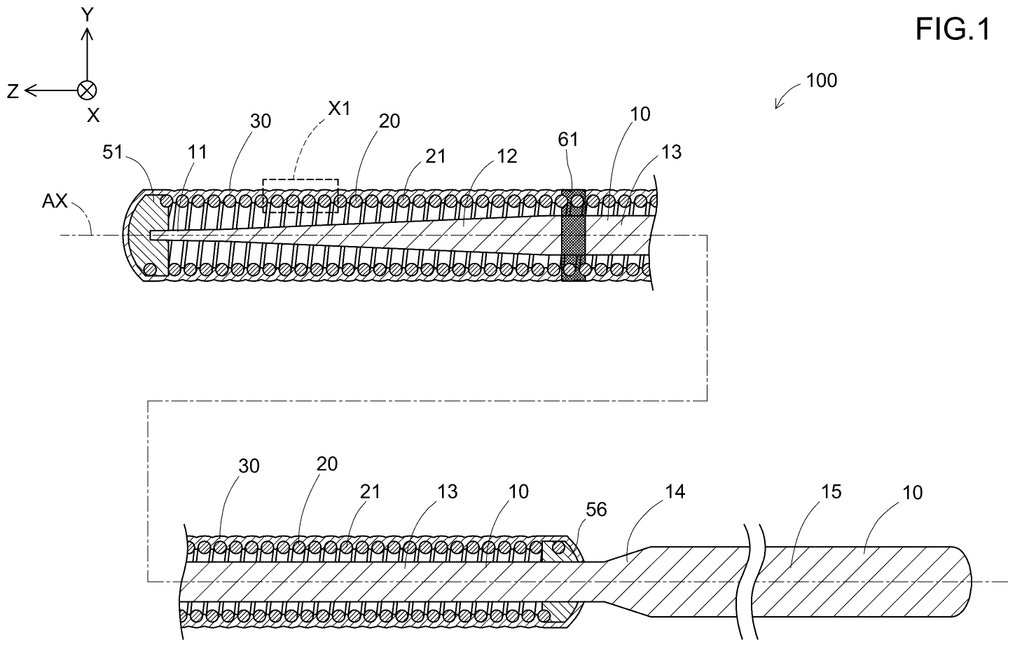

FIG. 1 is an explanatory diagram schematically illustrating a configuration of a guide wire 100 in the present embodiment. FIG. 1 illustrates the configuration of the guide wire 100 (and a core shaft 10 and a coil body 20 described later) in a longitudinal section (YZ cross-section) including a central axis AX. Note that depiction of the guide wire 100 is partially omitted in FIG. 1. In FIG. 1, the Z-axis positive direction side is a distal end side (far side) to be inserted into a body, and the Z-axis negative direction side is a proximal end side (near side) to be manipulated by a technician such as a surgeon. FIG. 1 illustrates a state where the entirety of the guide wire 100 is linear and substantially parallel to the Z-axis direction, but at least part of the guide wire 100 is configured to be flexible enough to be curved. In the present specification, with regard to the guide wire 100 and each component thereof, the end on the distal end side is referred to as a “distal end”, the distal end and the vicinity thereof are referred to as a “distal end portion”, the end on the proximal end side is referred to as a “proximal end”, and the proximal end and the vicinity thereof are referred to as a “proximal end portion”. The structure of a cross-section of each component in the guide wire 100 can be observed using, for example, a laser microscope.

The guide wire 100 is a long medical device inserted into a human body for guiding a combined device such as a catheter to a predetermined position inside a human body. The guide wire 100 has a total length of, for example, about 1500 mm to 3200 mm.

The guide wire 100 includes a core shaft 10, a coil body 20, a coating layer 30, a distal end side joint part 51, a proximal end side joint part 56, and an intermediate fixing part 61.

The core shaft 10 is a long member extending along the central axis AX. The core shaft 10 has a small diameter portion 11, a first tapered portion 12, a first large diameter portion 13, a second tapered portion 14, and a second large diameter portion 15 in the order from the distal end side toward the proximal end side. The small diameter portion 11 is a portion positioned at the most distal end of the core shaft 10, in which the outer diameter of the core shaft 10 is smallest. The first tapered portion 12 is a portion portioned between the small diameter portion 11 and the first large diameter portion 13 and having a tapered shape in which the outer diameter thereof increases from the distal end side toward the proximal end side. The first large diameter portion 13 is a portion portioned between the first tapered portion 12 and the second tapered portion 14 and having an outer diameter larger than the outer diameter of the small diameter portion 11. The second tapered portion 14 is a portion positioned between the first large diameter portion 13 and the second large diameter portion 15 and having a tapered shape in which the outer diameter thereof increases from the distal end side toward the proximal end side. The second large diameter portion 15 is a portion portioned at the most proximal end of the core shaft 10, in which the outer diameter of the core shaft 10 is largest. The second large diameter portion 15 is a portion held by a technician such as a surgeon. The shape of the transverse section (XY cross-section) at each position of the core shaft 10 may be any shape but is, for example, a circular shape or a flat-plate shape. The diameter and the length of each portion of the core shaft 10 can be arbitrarily set.

The core shaft 10 is formed from, for example, stainless steel (SUS302, SUS304, SUS316, etc.), a Ni—Ti alloy, a piano wire, a nickel-chromium-based alloy, a cobalt alloy, tungsten, or the like. The core shaft 10 may be formed from another superelastic alloy or another linear pseudo-elastic alloy.

The coil body 20 is a coiled member formed by helically winding a wire 21 to have a hollow cylindrical shape extending along the central axis AX. The coil body 20 is disposed on the outer periphery of the core shaft 10 so as to cover the core shaft 10. In the present embodiment, the coil body 20 covers the small diameter portion 11, the first tapered portion 12, and the first large diameter portion 13 of the core shaft 10. The wire 21 constituting the coil body 20 may be a solid wire composed of one strand or a twisted wire in which a plurality of strands is twisted. When the wire 21 is a solid wire, the coil body 20 is configured as a single coil, and when the wire 21 is a twisted wire, the coil body 20 is configured as a hollow twisted coil. The coil body 20 may be configured by combining a single coil and a hollow twisted coil. The wire diameter of the wire 21 and the average coil diameter (average diameter of the outer diameter and the inner diameter of the coil body 20) of the coil body 20 may be arbitrarily set. The coil body 20 is preferably a sparsely wound coil in which intervals are provided between coils of the wire 21 adjacent to each other in the axis direction. The coil body 20 may be a densely wound coil in which coils of the wire 21 adjacent to each other in the axis direction are close to each other.

The wire 21 constituting the coil body 20 is formed from, for example, a radiolucent material such as stainless steel (SUS302, SUS304, SUS316, etc.), a Ni—Ti alloy, or a piano wire; or a radiopaque material such as platinum, gold, tungsten, a cobalt alloy, a nickel-chromium-based alloy, or an alloy thereof. The wire 21 constituting the coil body 20 may be formed from another superelastic alloy or another linear pseudo-elastic alloy.

The distal end side joint part 51 is a member joining the distal end of the coil body 20 and the distal end (small diameter portion 11) of the core shaft 10. The proximal end side joint part 56 is a member joining the proximal end of the coil body 20 and the core shaft 10 (first large diameter portion 13). The intermediate fixing part 61 is a member joining the coil body 20 and the core shaft 10 (first large diameter portion 13) in the vicinity of the intermediate portion in the central axis AX direction of the coil body 20. The distal end side joint part 51, the proximal end side joint part 56, and the intermediate fixing part 61 are formed from, for example, metal solder (Au—Sn alloy, Sn—Ag alloy, Sn—Pb alloy, Pb—Ag alloy, etc.), a brazing material (aluminum alloy solder, silver solder, gold solder, etc.), an adhesive (epoxy-based adhesive, etc.), or the like. The materials forming the distal end side joint part 51, the proximal end side joint part 56, and the intermediate fixing part 61 may be identical or may differ from one another.

The coating layer 30 is a hydrophilic resin layer provided on the outer periphery of the coil body 20. The coating layer 30 covers at least an outer peripheral surface (outer surface) of the coil body 20. The coating layer 30 in the present embodiment also covers a surface on the distal end side of the distal end side joint part 51 and a surface on the proximal end side of the proximal end side joint part 56. The coating layer 30 may cover at least part of a surface of the second tapered portion 14 and/or the second large diameter portion 15 of the core shaft 10. The coating layer 30 improves lubricity of the guide wire 100 by absorbing moisture in a human body to swell.

### A-2. Detailed Configuration of Coating Layer 30:

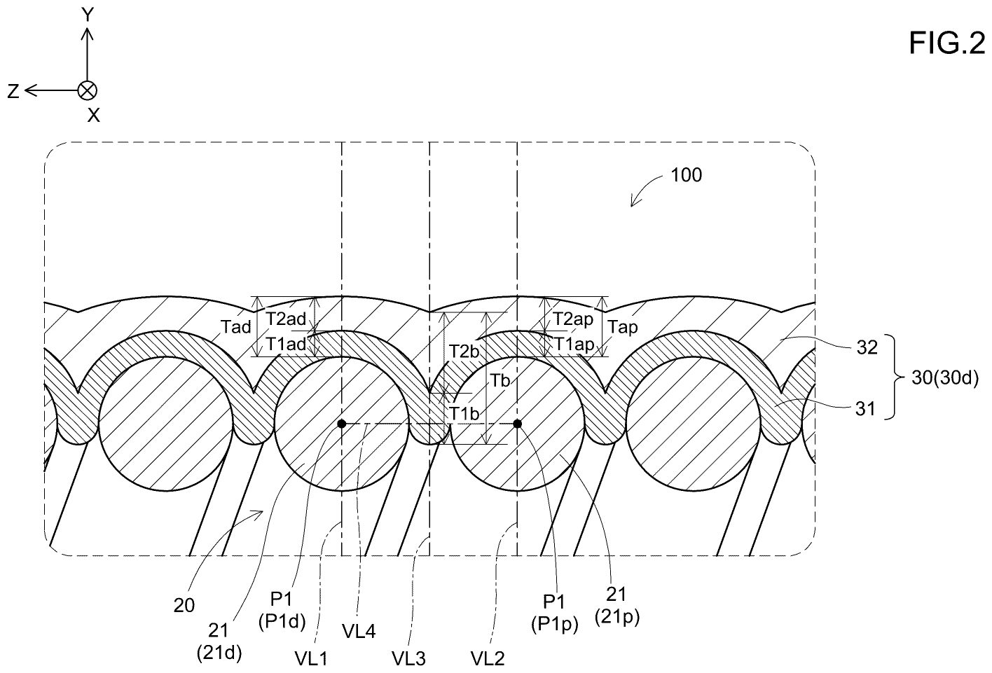

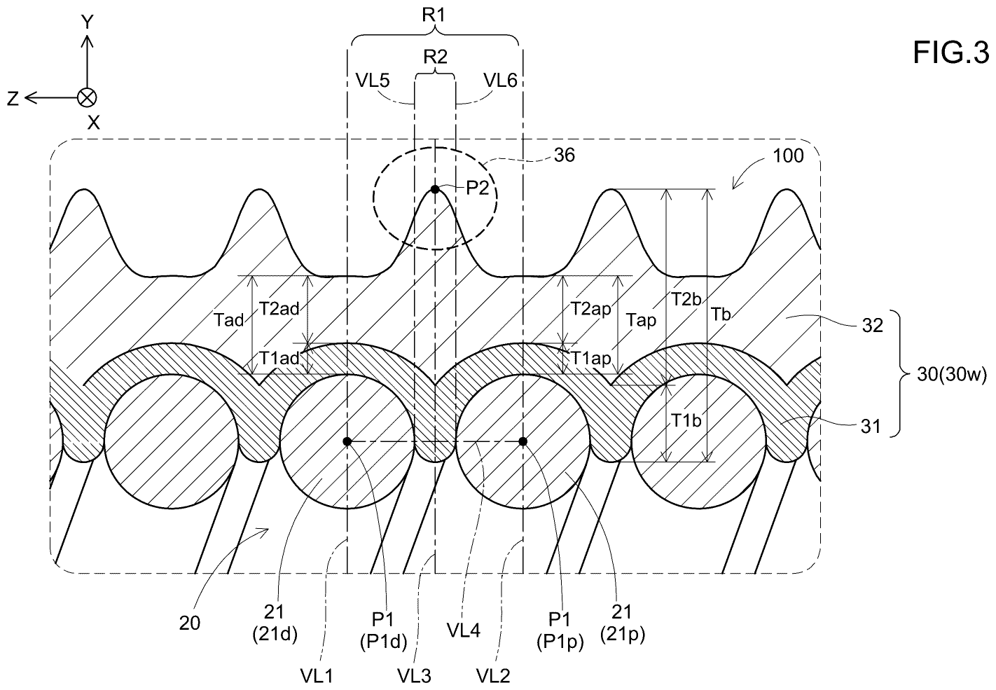

Next, a configuration of the coating layer 30 will be described in more detail. FIG. 2 and FIG. 3 are explanatory diagrams each illustrating a detailed configuration of the coating layer 30 of the present embodiment. FIG. 2 and FIG. 3 illustrate the configuration of a longitudinal section (YZ cross-section) of the coating layer 30 in the X1 portion in FIG. 1 in an enlarged manner. Multiple transverse sections of the wire 21 constituting the coil body 20 are present on the cross-sections illustrated in FIG. 2 and FIG. 3. FIG. 2 illustrates the configuration of the coating layer 30 in a normal state (dry state) (hereinafter, also referred to as the “coating layer 30d”), and FIG. 3 illustrates the configuration of the coating layer 30 in a swollen state (hereinafter, also referred to as the “coating layer 30w”). The swollen state herein means a state where the coating layer 30 is impregnated with physiological saline for 10 seconds or longer.

As illustrated in FIG. 2 and FIG. 3, the coating layer 30 has a two-layered structure including an inner coating layer 31 and an outer coating layer 32. The inner coating layer 31 is a layer provided on the outer periphery of the coil body 20. The outer coating layer 32 is a layer provided on the outer periphery of the inner coating layer 31. In the present embodiment, each of the inner coating layer 31 and the outer coating layer 32 is a continuous body the whole of which is continuously formed. Note that the inner coating layer 31 may be a discontinuous body a part of which is discontinuously formed. The inner coating layer 31 is an example of the first coating layer, and the outer coating layer 32 is an example of the second coating layer.

The outer coating layer 32 has swellability higher than that of the inner coating layer 31. High swellability of the coating layer herein means that the coating layer swells more (retains more water) when the coating layer is impregnated with physiological saline. A swollen film thickness, which is an amount of change in the film thickness measured using a laser microscope or the like can be used as an index value representing the level of swellability of the coating layer. The swollen film thickness of the outer coating layer 32 is preferably 1.2 times or more, more preferably 1.5 times or more, and still more preferably 2.0 times or more the swollen film thickness of the inner coating layer 31. The swollen film thickness of the coating layer can be measured using VFX series (manufactured by KEYENCE CORPORATION), which are laser microscopes, OPTELICS series (manufactured by Lasertec Corporation), which are white confocal microscopes, and F40 series (manufactured by Filmetrics, INC.), which are optical interference film thickness meters. Especially, in a case where VFX-8710, which is a laser microscope, is used, the swollen film thickness can be measured as follows: the guide wire 100, in a dry state, provided with a predetermined hydrophilic coating is observed, the dry film thickness from the coil body 20 to the surface of the coating layer is measured, and physiological saline is then dropped onto a portion to be observed to cause the coil body 20 and the coating layer 30 to be immersed in physiological saline for seconds or longer, and the coil body 20 and the coating layer 30 are then further measured with the laser microscope.

For example, as a material for forming the inner coating layer 31, polyvinyl alcohol (PVA), hydrophilic urethane resin (for example, Hydro Thane (Mitsubishi Chemical Corporation), Hydro MED (Mitsubishi Chemical Corporation), Bionate (DSM), Tecophilic (Lubrizol), HPU (Dainichiseika Color & Chemicals Mfg. Co., Ltd.)), modified polyolefin resin (for example, polyethylene-acrylic acid (UNITIKA LTD.), BONDINE (TOKYO ZAIRYO CO., LTD.)), and the like can be used, and polyvinyl alcohol (PVA) and hydrophilic urethane resin (Hydro Thane) can be especially preferably used. As a material for forming the outer coating layer 32, hyaluronic acid, carboxybetaine, phosphobetaine, sulfobetaine, polyvinylpyrrolidone, maleic acid, acrylic acid, methacrylic acid, dimethylacrylamide, methoxyethyl acrylate, hydroxyethyl methacrylate, 2-hydroxypropyl methacrylate, polyethylene glycol, copolymers thereof, and the like can be used, and hyaluronic acid, carboxybetaine, phosphobetaine, dimethylacrylamide, and copolymers thereof can be more preferably used. When the inner coating layer 31 and the outer coating layer 32 are formed using these materials, swellability of the outer coating layer 32 can be made higher than that of the inner coating layer 31.

As illustrated in FIG. 2, in the present embodiment, the thickness of the outer coating layer 32 at the position between coils of the wire 21 is thicker than that at the position immediately above the wire 21 of the coil body 20, in the coating layer 30 (30d) in the normal state (dry state). More specifically, on the cross-section (cross-section illustrated in FIG. 2) including the central axis AX of the coil body 20, a first thickness T2b of the outer coating layer 32 on the perpendicular bisector VL3 of a virtual line segment VL4 connecting the central point P1 (hereinafter, referred to as the “first central point P1d”) of one (certain one) transverse section (“a first transverse section”) 21d of the wire 21 of the coil body 20 and the central point P1 (hereinafter, referred to as the “second central point P1p”) of another transverse section (“a second transverse section”) 21p of the wire 21 is thicker than a second thickness T2ad of the outer coating layer 32 on a first virtual straight line VL1 passing the first central point P1d and perpendicular to the central axis AX and is thicker than a third thickness T2ap of the outer coating layer 32 on a second virtual straight line VL2 passing the second central point P1p and perpendicular to the central axis AX, the second transverse section 21p being adjacent to and on the proximal end side of the first transverse section 21d. Note that in the present embodiment, such relation between thicknesses is established for an arbitrary pair of transverse sections of the wire 21 adjacent to each other on an arbitrary cross-section including the central axis AX of the coil body 20. Since the coil body 20 has the configuration in which the wire 21 is helically wound, in the coating layer 30 (30d) in the normal state, the portion in which the thickness of the outer coating layer 32 is relatively thick is helically distributed along the region between coils of the wire 21.

On the cross-section including the central axis AX of the coil body 20 in the coating layer 30 (30d) in the normal state, the first thickness T2b of the outer coating layer 32 on the perpendicular bisector VL3 is preferably 1.2 times or more the second thickness T2ad of the outer coating layer 32 on the first virtual straight line VL1 and is 1.2 times or more the third thickness T2ap of the outer coating layer 32 on the second virtual straight line VL2, and is more preferably 2 times or more the thickness T2ad and 2 times or more the thickness T2ap. In addition, the first thickness T2b of the outer coating layer 32 on the perpendicular bisector VL3 is preferably 5 times or less the second thickness T2ad of the outer coating layer 32 on the first virtual straight line VL1 and 5 times or less the third thickness T2ap of the outer coating layer 32 on the second virtual straight line VL2, and is more preferably 3.5 times or less the second thickness T2ad and 3.5 times or less the third thickness T2ap. The first thickness T2b of the outer coating layer 32 on the perpendicular bisector VL3 is, for example, about 1.5 μm to 4.0 μm, and the second thickness T2ad of the outer coating layer 32 on the first virtual straight line VL1 and the third thickness T2ap of the outer coating layer 32 on the second virtual straight line VL2 are, for example, about 0.5 μm to 2.0 μm. The second thickness T2ad and the third thickness T2ap may be the same or different.

In the present embodiment, in the coating layer 30 (30d) in the normal state, the thickness of the inner coating layer 31 at the position between coils of the wire 21 is thicker than that at the position immediately above the wire 21 of the coil body 20. More specifically, on the cross-section (cross-section illustrated in FIG. 2) including the central axis AX of the coil body 20, the thickness T1b of the inner coating layer 31 on the perpendicular bisector VL3 is thicker than the thickness T1ad of the inner coating layer 31 on the first virtual straight line VL1 and is thicker than the thickness T1ap of the inner coating layer 31 on the second virtual straight line VL2. In addition, in the present embodiment, in the coating layer 30 (30d) in the normal state, the thickness of the coating layer 30 as a whole at the position between coils of the wire 21 is thicker than that at the position immediately above the wire 21 of the coil body 20. More specifically, on the cross-section including the central axis AX of the coil body 20, the thickness Tb of the coating layer 30 on the perpendicular bisector VL3 is thicker than the thickness Tad of the coating layer 30 on the first virtual straight line VL1 and is thicker than the thickness Tap of the coating layer 30 on the second virtual straight line VL2.

When the coating layer 30 is shifted to the swollen state from the normal state, as illustrated in FIG. 3, each layer constituting the coating layer 30 absorbs moisture and swells. Since the outer coating layer 32 has swellability higher than that of the inner coating layer 31, the swelling amount of the outer coating layer 32 is larger than the swelling amount of the inner coating layer 31. Since the coating layer 30 in the present embodiment has the above-described configuration (configuration in which the thickness of the outer coating layer 32 with relatively high swellability is relatively thick at the portion between coils of the wire 21 of the coil body 20) in the normal state, when the normal state is shifted to the swollen state, the portion in which the thickness of the outer coating layer 32 is relatively thick in the normal state largely swells to form a peak portion 36. That is, the coating layer 30 (30w) is configured to have the peak portion 36 protruding toward the outer peripheral direction of the guide wire 100 in the swollen state.

An apex P2 of the peak portion 36 is not positioned immediately above the wire 21 of the coil body 20 but positioned between coils of the wire 21. More specifically, on the cross-section (cross-section illustrated in FIG. 3) including the central axis AX of the coil body 20, the apex P2 of the peak portion 36 is present at a position on the proximal end side of the first virtual straight line VL1 and at a position on the distal end side of the second virtual straight line VL2 (range R1 in FIG. 3). In other words, the apex P2 of the peak portion 36 is positioned neither on the first virtual straight line VL1 nor on the second virtual straight line VL2 but is present at a position other than positions on the first virtual straight line VL1 and the second virtual straight line VL2. In the present embodiment, the relationship pertaining to the position of the apex P2 of the peak portion 36 is established for an arbitrary pair of transverse sections of the wire 21 adjacent to each other on an arbitrary cross-section including the central axis AX of the coil body 20. As described above, since the coil body 20 has the configuration in which the wire 21 is helically wound, and the portion in which the thickness of the outer coating layer 32 is relatively thick is helically distributed along the region between coils of the wire 21 in the coating layer 30 (30d) in the normal state, the peak portion 36 is continuously formed such that the apex P2 thereof helically extends along the region between coils of the wire 21 in the coating layer 30 (30w) in the swollen state.

In the present embodiment, on the cross-section including the central axis AX of the coil body 20 in the coating layer 30 (30w) in the swollen state, the apex P2 of the peak portion 36 is present at a position not overlapping the transverse sections of the wire 21 in the direction (in the example of FIG. 3, Y-axis direction) perpendicular to the central axis AX. In other words, the apex P2 of the peak portion 36 is present at a position on the proximal end side of a fifth virtual straight line VL5 passing through the end point on the proximal end side of first transverse section 21d of the wire 21 of the coil body 20 and perpendicular to the central axis AX and at a position on the distal end side of a sixth virtual straight line VL6 passing through the end point on the distal end side of second transverse section 21p and perpendicular to the central axis AX (range R2 in FIG. 3), the second transverse section 21p being adjacent to and on the proximal end side of the first transverse section 21d. More specifically, in the present embodiment, on the cross-section including the central axis AX of the coil body 20 in the coating layer 30 (30w) in the swollen state, the apex P2 of the peak portion 36 is positioned on the perpendicular bisector VL3 or in the immediate vicinity thereof. Since the peak portion 36 is a portion protruding in the outer peripheral direction of the guide wire 100, the apex 2P of the peak portion 36 is positioned on the more outer peripheral side than the virtual line segment VL4.

On the cross-section including the central axis AX of the coil body 20 in the coating layer 30 (30w) in the swollen state, the thickness T2b of the outer coating layer 32 at the position of the apex P2 of the peak portion 36 is, for example, about 4.0 μm to 16.0 μm, and the thickness T2ad of the outer coating layer 32 on the first virtual straight line VL1 and the thickness T2ap of the outer coating layer 32 on the second virtual straight line VL2 are, for example, about 1.0 μm to 6.0 μm.

### A-3. Guide Wire 100 Production Method:

A method of producing a guide wire 100 of the present embodiment is, for example, as follows. First, a coil body 20 is joined to a core shaft 10 by a joint part (a distal end side joint part 51, a proximal end side joint part 56, and an intermediate fixing part 61) to prepare a guide wire 100 before forming a coating layer 30. The guide wire 100 is cleaned, if needed.

Next, an inner coating layer 31 with relatively low swellability is formed for the guide wire 100 by a predetermined film forming method. For example, a solution for the inner coating layer 31 is prepared using a resin material with relatively low swellability, and the inner coating layer 31 is formed by dip coating using the solution. Then, an outer coating layer 32 with relatively high swellability is formed, by a predetermined film forming method, for the guide wire 100 with the inner coating layer 31 formed therein. For example, a solution for the outer coating layer 32 is prepared using a resin material with relatively high swellability, and the outer coating layer 32 is formed by dip coating using the solution. The guide wire 100 provided with a coating layer 30 composed of the inner coating layer 31 and the outer coating layer 32 can be produced by the above method.

When the outer coating layer 32 is formed, the thickness of the outer coating layer 32 at a position between coils of the wire 21 is thicker than that at a position immediately above the wire 21 of the coil body 20. A configuration in which the apex P2 of the peak portion 36 of the coating layer 30 is positioned immediately above the wire 21 of the coil body 20 but positioned between coils of the wire 21 when the coating layer 30 is shifted from the normal state (dry state) to the swollen state can be realized in this manner. Adjustment of the extent to which the peak portion 36 of the coating layer 30 protrudes in the swollen state can be achieved by, for example, adjusting the thicknesses of the outer coating layer 32 and the inner coating layer 31 or by adjusting the distance between coils of the wire 21 of the coil body 20.

### A-4. Function and Effect of Guide Wire 100:

The guide wire 100 of the present embodiment has the above-described configuration and thus can achieve both lubricity and passability of a combined device at a high level. Hereinafter, this point will be described.

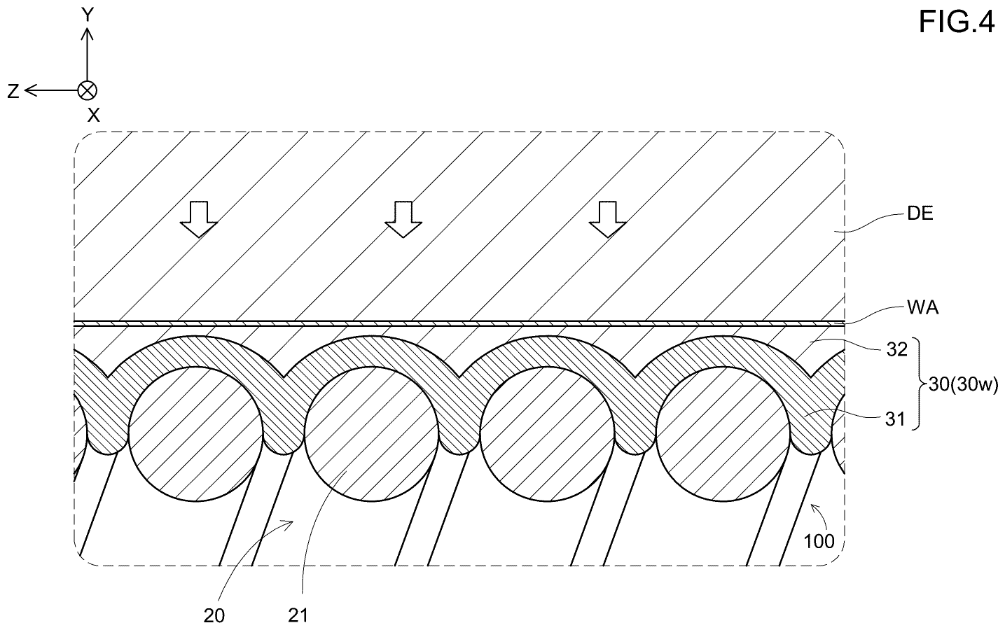

FIG. 4 is an explanatory diagram illustrating a state of the guide wire 100 of the present embodiment during use. When the guide wire 100 is inserted into a human body, the coating layer 30 absorbs moisture and swells (see FIG. 3). Thereafter, a combined device DE such as a balloon catheter is inserted into the human body while being guided by the guide wire 100. At that time, for example, when the combined device DE is pressed against the coating layer 30 (30w) in a bent portion or the like of the combined device DE, as illustrated in FIG. 4, the coating layer 30 is crushed to have a smooth surface, and moisture is extruded from the coating layer 30 to form a thin moisture membrane WA between the smooth surface of the coating layer 30 and the combined device DE; as a result, lubricity between the guide wire 100 and the combined device DE is ensured.

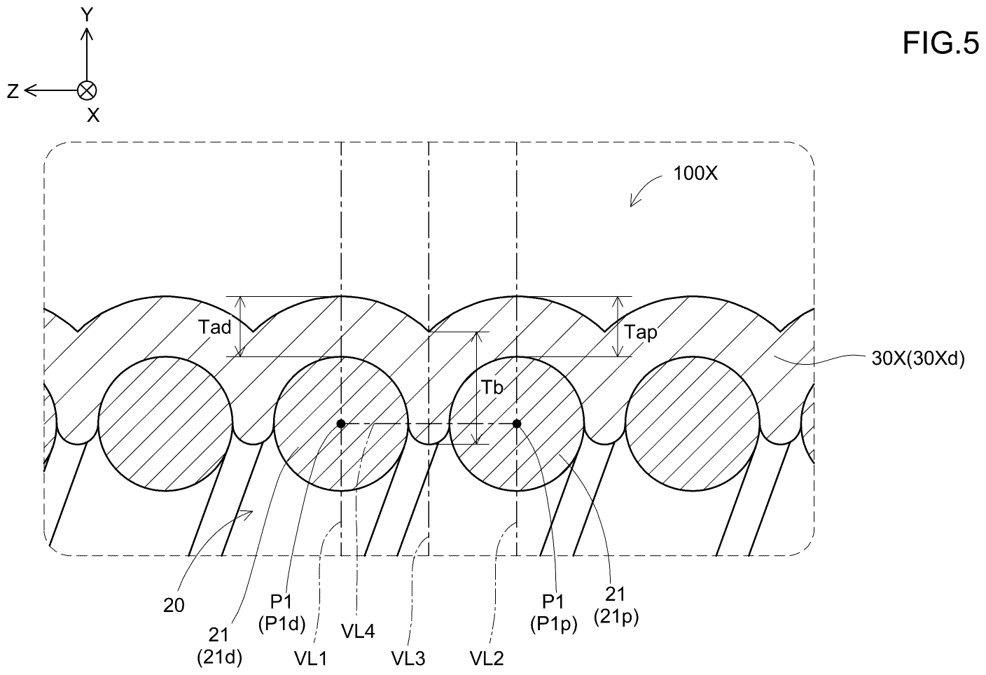

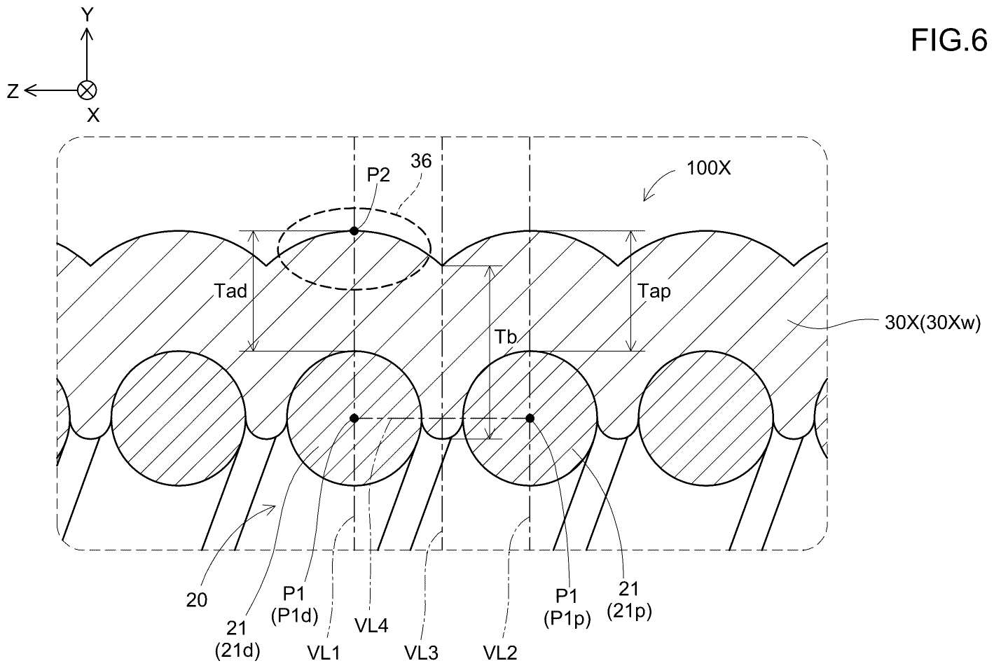

FIG. 5 and FIG. 6 are explanatory diagrams each illustrating a configuration of a coating layer 30X in a guide wire 100X of a comparative example. FIG. 5 illustrates the configuration of the coating layer 30X (30Xd) in a normal state (dry state), and FIG. 6 illustrates the configuration of the coating layer 30X (30Xw) in a swollen state.

As illustrated in FIG. 5 and FIG. 6, the coating layer 30X of the comparative example is composed of a single layer. The swellability of the coating layer 30X of the comparative example is approximately identical to the swellability of the outer coating layer 32 constituting the coating layer 30 of the above-described embodiment. The thickness of the coating layer 30X of the comparative example in the normal state is approximately constant at every position. Therefore, the shape of the outer peripheral surface of the coating layer 30X is similar to the shape of the outer peripheral surface of the wire 21 of the coil body 20. However, because the coating layer 30X penetrates the gap between coils of the wire 21 of the coil body 20, the thickness Tb of the coating layer 30X on the perpendicular bisector VL3 is slightly thicker than the thickness Tad of the coating layer 30X on the first virtual straight line VL1 and slightly thicker than the thickness Tap of the coating layer 30X on the second virtual straight line VL2. The thicknesses Tad, Tap of the coating layer 30X of the comparative example on the first virtual straight line VL1 and on the second virtual straight line VL2 are approximately identical to the thicknesses Tad, Tap of the coating layer 30 of the above-described embodiment at the positions on the first virtual straight line VL1 and on the second virtual straight line VL2.

As the coating layer 30X of the comparative example has such a configuration in the normal state, when the normal state is shifted to the swollen state, a peak portion 36 protruding in the outer peripheral direction of the guide wire 100X is formed as illustrated in FIG. 6. However, the coating layer 30X of the comparative example has a single layer structure, and the thickness thereof is approximately constant; therefore, an apex P2 of the peak portion 36 is positioned immediately above the wire 21 of the coil body 20. That is, the apex P2 of the peak portion 36 is positioned on the first virtual straight line VL1 and on the second virtual straight line VL2. In the swollen state, the thicknesses Tad, Tap at the position of the apex P2 of the peak portion 36 of the coating layer 30X (30Xw) of the comparative example are thinner than the thickness Tb at the position of the apex P2 of the peak portion 36 of the coating layer 30 (30w) of the above-described embodiment.

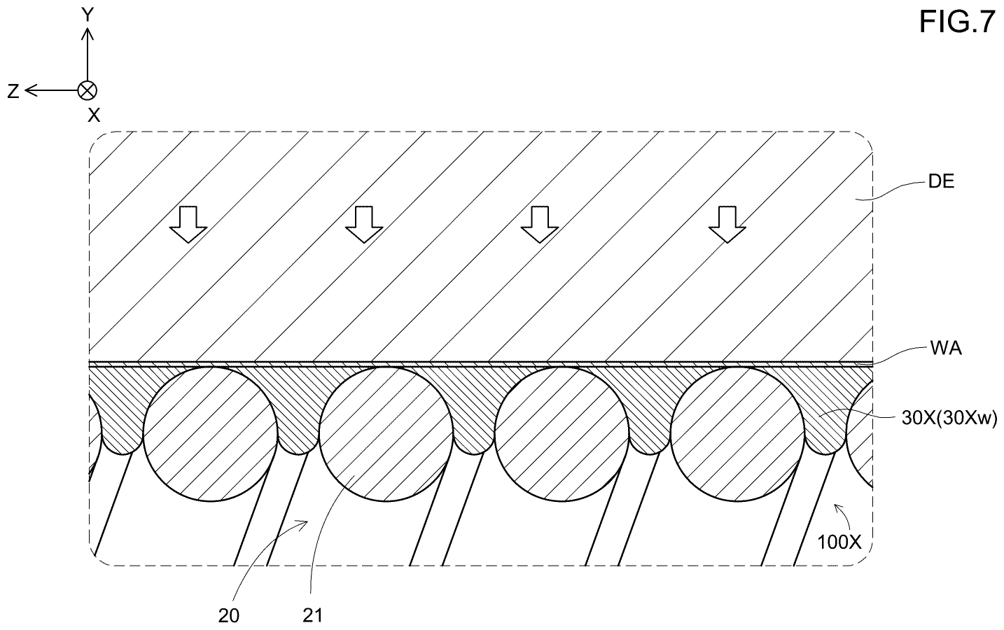

FIG. 7 is an explanatory diagram illustrating a state of the guide wire 100X of the comparative example during use. Similar to the above-described embodiment, when the guide wire 100X is inserted into a human body, the coating layer 30X absorbs moisture and swells also in the comparative example (see FIG. 6). Thereafter, a combined device DE is inserted into the human body while being guided by the guide wire 100X. At that time, for example, when the combined device DE is pressed against the coating layer 30X (30Xw) in a bent portion or the like of the combined device DE, the coating layer 30X is crushed as illustrated in FIG. 7.

In the comparative example, the apex P2 of the peak portion 36 of the coating layer 30X (30Xw) in the swollen state is positioned immediately above the wire 21 of the coil body 20 as illustrated in FIG. 6. Since the wire 21 functions as a base in the portion of the coating layer 30X positioned immediately above the wire 21, the portion of the coating layer 30X positioned immediately above the wire 21 has small “allowance” when receiving stress from the outer peripheral side and thus cannot flexibly receive stress from the combined device DE. As a result, in the comparative example, the coating layer 30X is crushed and the combined device DE comes into contact with the wire 21 of the coil body 20 as illustrated in FIG. 7, possibly causing decrease in lubricity.

In the comparative example, it is also considered that the thickness of the coating layer 30X is thickened so as to avoid contact between the combined device DE and the wire 21 of the coil body 20. However, when the thickness of the coating layer 30X is simply thickened, the thickness of the portion (peak portion 36) positioned immediately above the wire 21 of the coil body 20 in the coating layer 30X becomes excessively large in the swollen state, possibly causing deterioration in passability of the combined device DE.

On the other hand, in the guide wire 100 of the present embodiment, the coating layer 30 is configured to have the peak portion 36 protruding in the outer peripheral direction of the guide wire 100 in the swollen state. Therefore, the peak portion 36 in the coating layer 30 effectively exhibits a water retention function, and high lubricity is imparted to the guide wire 100.

In the guide wire 100 of the present embodiment, on the cross-section including the central axis AX of the coil body 20, the apex P2 of the peak portion 36 of the coating layer 30 in the swollen state is present at a position on the proximal end side of the first virtual straight line VL1 passing through the first central point P1d of the first transverse section 21d of the wire 21 of the coil body 20 and perpendicular to the central axis AX and at a position on the distal end side of the second virtual straight line VL2 passing through the second central point P1p of the second transverse section 21p of the wire 21 of the coil body 20 and perpendicular to the central axis AX, the second transverse section 21p being adjacent to and on the proximal end side of the first transverse section 21d. That is, the apex P2 of the peak portion 36 of the coating layer 30 in the swollen state is present between coils of the wire 21 of the coil body 20. The portion of the coating layer positioned between coils of the wire 21 is a portion less affected by the wire 21 as a base and thus is a portion with large “allowance” when receiving stress from the outer peripheral side. Therefore, when the apex P2 of the peak portion 36 is present in the above-described portion of the coating layer 30 (in other words, when the volume of the coating layer 30 is large in the above-described portion), stress from the combined device DE can be dispersed. Accordingly, according to the guide wire 100 of the present embodiment, the combined device DE is effectively prevented from coming into contact with the wire 21 of the coil body 20 even when the coating layer 30 is crushed.

Since the presence of the wire 21 as a base is minor in the portion of the coating layer 30 positioned between coils of the wire 21, the portion of the coating layer 30 positioned between coils of the wire 21 easily moves, and passability of the combined device DE is thus successfully maintained even when the apex P2 of the peak portion 36 of the coating layer 30 is positioned between coils of the wire 21 of the coil body 20.

In view of the above, according to the guide wire 100 of the present embodiment, both lubricity and passability of the combined device DE can be achieved at a high level.

In the present embodiment, on the cross-section including the central axis AX of the coil body 20, the apex P2 of the peak portion 36 is present at a position not overlapping the transverse sections of the wire 21 of the coil body 20 in the direction perpendicular to the central axis AX. The portion of the coating layer 30 present at a position not overlapping the transverse sections of the wire 21 of the coil body 20 in the direction perpendicular to the central axis AX is a portion significantly less affected by the wire 21 as a base and thus is a portion with significantly large “allowance” when receiving stress from the outer peripheral side. Therefore, when the apex P2 of the peak portion 36 is present in the above-described portion of the coating layer 30, stress from the combined device DE can be effectively dispersed. Accordingly, according to the guide wire 100 of the present embodiment, the combined device DE is more effectively prevented from coming into contact with the wire 21 of the coil body 20 even when the coating layer 30 is crushed, and lubricity of the guide wire 100 can be more effectively improved.

In the guide wire 100 of the present embodiment, the coating layer 30 includes the inner coating layer 31 that is provided on the outer periphery of the coil body 20 and the outer coating layer 32 that is provided on the outer periphery of the inner coating layer 31 and has swellability higher than that of the inner coating layer 31. In the normal state, on the cross-section including the central axis AX of the coil body 20, the thickness T2b of the outer coating layer 32 on the perpendicular bisector VL3 is thicker than the thickness T2ad of the outer coating layer 32 on the first virtual straight line VL1 and thicker than the thickness T2ap of the outer coating layer 32 on the second virtual straight line VL2. As the guide wire 100 of the present embodiment has such a configuration, the configuration in which the apex P2 of the peak portion 36 of the coating layer 30 in the swollen state is present at a position on the more proximal end side than the first virtual straight line VL1 and at a position on the more distal end side than the second virtual straight line VL2 can be realized. Accordingly, according to the guide wire 100 of the present embodiment, as described above, both lubricity and passability of the combined device DE can be achieved at a high level.

In the normal state, on the cross-section including the central axis AX of the coil body 20, the thickness T2b of the outer coating layer 32 on the perpendicular bisector VL3 is preferably 1.2 times or more the thickness T2ad of the outer coating layer 32 on the first virtual straight line VL1 and 1.2 times or more the thickness T2ap of the outer coating layer 32 on the second virtual straight line VL2. With such a configuration, the configuration in which in the coating layer 30 in a swollen state, the thickness of the portion positioned between coils of the wire 21 of the coil body 20 is thicker than the thickness of the portion positioned immediately above the wire 21 by a certain factor or more can be realized, and both lubricity and passability of the combined device DE can be achieved at a higher level.

### B. Modifications:

The technology disclosed in the present specification is not limited to the embodiment described above and may be modified into various aspects without departing from the spirit thereof and may be modified as described below, for example.

The configuration of the guide wire 100 according to the above-described embodiment is merely an example and may be modified in various manners. For example, although the coating layer 30 has a two layer structure with the inner coating layer 31 and the outer coating layer 32 in the above-described embodiment, the coating layer 30 may have a structure with three or more layers in which one or more other layers are disposed between the inner coating layer 31 and the outer coating layer 32.

Although, on the cross-section including the central axis AX of the coil body 20 in the coating layer 30 (30w) in the swollen state, the apex P2 of the peak portion 36 is positioned on the perpendicular bisector VL3 or in the immediate vicinity thereof in the above-described embodiment, the apex P2 of the peak portion 36 may be present at another position as long as the position is neither on the first virtual straight line VL1 nor on the second virtual straight line VL2.

Although, the peak portion 36 is helically and continuously formed in the coating layer 30 (30w) in the swollen state, in the above-described embodiment, the peak portion 36 may be discretely formed.

Although the relationship in which the apex P2 of the peak portion 36 is present at a position on the more proximal end side than the first virtual straight line VL1 and at a position on the more distal end side than the second virtual straight line VL2 is established for an arbitrary pair of transverse sections of the wire 21 adjacent to each other on an arbitrary cross-section including the central axis AX of the coil body 20 in the above-described embodiment, the relationship may be established for at least one pair of transverse sections of the wire 21 adjacent to each other on at least one cross-section including the central axis AX of the coil body 20. Similarly, the relationship in which the thickness T2b of the outer coating layer 32 on the perpendicular bisector VL3 is thicker than the thickness T2ad of the outer coating layer 32 on the first virtual straight line VL1 and thicker than the thickness T2ap of the outer coating layer 32 on the second virtual straight line VL2 is established for an arbitrary pair of transverse sections of the wire 21 adjacent to each other on an arbitrary cross-section including the central axis AX of the coil body 20 in the above-described embodiment, the relationship may be established for at least one pair of transverse sections of the wire 21 adjacent to each other on at least one cross-section including the central axis AX of the coil body 20.

Hereinafter, an example of disclosed embodiments will be described for more clearly describing the contents of the disclosed embodiments described above.

### EXAMPLE 1

First, a guide wire 100 (guide wire 100 before forming a coating layer 30) including a core shaft 10, a coil body 20, a distal end side joint part 51, a proximal end side joint part 56, and an intermediate fixing part 61 was prepared, and a surface of the guide wire 100 was scraped using unwoven fabric into which isopropyl alcohol (IPA) was penetrated to clean same.

Next, polyvinyl alcohol (molecular weight: 2600, saponification degree: 98% or more, product name: NH26-S manufactured by Mitsubishi Chemical Corporation) was dissolved in hot water at a concentration of 5%, and 0.16% of polycarbodiimide (manufactured by Nisshinbo Holdings Inc., product name: Carbodilite) was subsequently added as a cros slinking agent to obtain a solution for an inner coating layer 31. Dip coating was conducted on the guide wire 100 before forming a coating layer 30 using the obtained solution, followed by heating and drying at 70° C. for one hour to obtain a guide wire 100 with an inner coating layer 31 formed therein.

Sodium hyaluronate (molecular weight: about one million) was dissolved in a mixture solution (water: N-methylpyrrolidone=85:15) at a concentration of 0.8 wt % to obtain a solution for an outer coating layer 32. Dip coating was conducted on the guide wire 100 with the inner coating layer 31 formed therein using the obtained solution, followed by heating and drying at 120° C. for one hour to form an outer coating layer 32. A guide wire 100 provided with a coating layer 30 composed of the inner coating layer 31 and the outer coating layer 32 was obtained thereby.

Film shape Evaluation Method

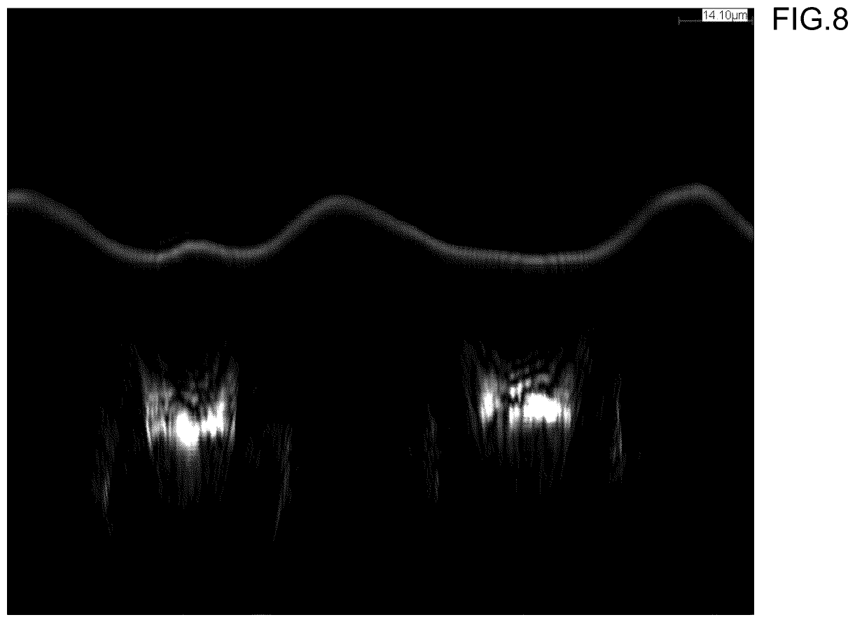

As measurement of a swollen film thickness, after immersing, in physiological saline for 20 seconds, the guide wire 100 of Example 1 with the coating layer 30 formed therein, the transparent film cross-section was measured with a laser using a laser microscope (VFX-8710, manufactured by KEYENCE CORPORATION). FIG. 8 is an explanatory diagram illustrating a swollen film thickness measurement result of the guide wire 100 of Example 1. In FIG. 8, the whitest portion is a portion with the largest laser reflection and corresponds to the outermost surface of each coil of the wire 21 constituting the coil body 20. The portion above the whitest portion, observed as being corrugated is the coating layer 30 (30w) swollen with physiological saline. In the coating layer 30 (30w), the peak portion 36 upwardly protruding is formed at an intermediate position of the outermost surface (the portion with the largest reflection) of each coil of the wire 21 of the coil body 20. In Example 1 illustrated in FIG. 8, the thickness Ta of the coating layer 30 (30w) at a position immediately above the wire 21 of the coil body 20 is 5.80 μm, and the thickness Tb of the coating layer 30 (30w) at the position (position between coils of the wire 21 of the coil body 20) of the peak portion 36 is 12.40 μm.

### Lubricity Evaluation Method

A portion of a balloon catheter (Kamui 3.00 mm×15 mm, manufactured by ASAHI INTECC CO., LTD.), which is a combined device for the guide wire 100, on the proximal end side from 105 mm from the distal end side is wound once around a column with an outer diameter of 30 mm to reproduce a bent portion of a coronary artery. The guide wire 100 of Example 1 was inserted into the balloon catheter in this state, and lubricity at that time was evaluated. As a result, it was demonstrated that resistance during insertion of the guide wire 100 of Example 1 was significantly small, and high lubricity was exhibited.

As a more severe test, the guide wire 100 was repeatedly inserted into and removed from the balloon catheter 50 times, and a change in lubricity was evaluated. As a result, no change in the resistance value was found even after repeating insertion and removal 50 times in Example 1, and high lubricity was maintained.

From the above observation result and the above evaluation result, it was observed that when the peak portion 36 of the coating layer 30 (30w) was pressed against the balloon catheter, which was the combined device, at the position between coils of the wire 21 of the coil body 20 in the guide wire 100, the peak portion 36 deformed to provide a moisture membrane to the apex of the wire 21 of the coil body 20, and high lubricity was thus maintained.

The disclosed embodiments are not limited to the conditions of the above example, and various base materials and coating agents can be selected without departing from the spirit of the disclosed embodiments.

### DESCRIPTION OF REFERENCE NUMERALS

10 Core shaft
11 Small diameter portion
12 First tapered portion
13 First large diameter portion
14 Second tapered portion
15 Second large diameter portion
20 Coil body
21 Wire
30 Coating layer
31 Inner coating layer
32 Outer coating layer
36 Peak portion
51 Distal end side joint part
56 Proximal end side joint part
61 Intermediate fixing part
100 Guide wire
AX Central axis
DE Combined device
WA Membrane

1. A guide wire comprising:
a core shaft;
a coil body including a wire helically wound around an outer periphery of the core shaft; and
a coating layer provided on an outer periphery of the coil body, wherein
the coating layer includes a peak portion protruding in an outer peripheral direction of the guide wire in a swollen state,
in a cross-section along a central axis of the coil body, an apex of the peak portion is present at a position on:
a proximal end side of a first virtual straight line that extends through a central point of a first transverse section of the wire and is perpendicular to the central axis, and
a distal end side of a second virtual straight line that extends through a central point of a second transverse section of the wire and is perpendicular to the central axis, and
the second transverse section is adjacent to and on a proximal end side of the first transverse section.

2. The guide wire according to claim 1, wherein
in the cross-section along the central axis of the coil body, the apex of the peak portion is present at a position that does not overlap transverse sections of the wire in a direction perpendicular to the central axis.

3. A guide wire comprising:
a core shaft;
a coil body including a wire helically wound around an outer periphery of the core shaft; and
a coating layer provided on an outer periphery of the coil body, wherein
the coating layer includes a first coating layer provided on the outer periphery of the coil body, and a second coating layer provided on an outer periphery of the first coating layer and having swellability higher than swellability of the first coating layer,
in a cross-section along a central axis of the coil body, the second coating layer has:
a first thickness at a position of a perpendicular bisector of a virtual line segment connecting a central point of a first transverse section of the wire and a central point of a second transverse section of the wire,
a second thickness at a position of a first virtual straight line that extends through the central point of the first transverse section and is perpendicular to the central axis, and
a third thickness at a position of a second virtual straight line that extends through the central point of the second transverse section and is perpendicular to the central axis,
the first thickness is thicker than each of the second thickness and the third thickness, and
the second transverse section is adjacent to and on a proximal end side of the first transverse section.

4. The guide wire according to claim 3, wherein
in the cross-section along the central axis of the coil body, the first thickness of the second coating layer is 1.2 times or more the second thickness and is 1.2 times or more the third thickness.
